(19) United States
(12) Patent Application Publication
(10) Pub. No.: US 2020/0000470 A1
(43) Pub. Date: Jan. 2, 2020

(54) ANVIL PLATE FOR A SURGICAL STAPLING INSTRUMENT

(71) Applicant: Covidien LP, Mansfield, MA (US)

(72) Inventors: Fen Du, Beijing (CN); Jiangfeng Zhang, Shanghai (CN); Hui Zhan, Shanghai (CN)

(21) Appl. No.: 16/486,190

(22) PCT Filed: Mar. 17, 2017
(86) PCT No.: PCT/CN2017/077107

§ 371 Date: Aug. 15, 2019

(51) Int. Cl. A61B 17/072

(52) U.S. Cl. CPC A61B 17/07207; A61B 2017/00398; A61B 2017/07271; A61B 2017/07257

(57) ABSTRACT

An anvil for use with a linear stapling instrument includes an anvil body defining a longitudinal axis and an anvil plate. The anvil plate includes a base having a first side defining at least one staple forming pocket and a second side having a pair of outer beams extending along the longitudinal axis and a central beam disposed between the outer beams. The central beam includes at least one mounting tab, which is receivable within at least one corresponding tab receiving opening of the anvil body to mount the anvil plate relative to the anvil body.

### BACKGROUND

### 1. Technical Description

The present disclosure relates to a surgical stapling instrument, and, more particularly, relates to an anvil plate for use with a linear stapling instrument.

### 2. Background of Related Art

Surgical stapling instruments for dispensing staples to join tissue during surgical procedures are well known. Linear type surgical stapling instruments typically include a staple cartridge housing a plurality of staples and an anvil disposed in opposition to the staple cartridge. Tissue is disposed between the staple cartridge and the anvil, and these components are approximated. A firing mechanism is activated to sequentially eject staples from the staple cartridge through the tissue for deformation by the anvil. A knife may be utilized to sever tissue between sets of the applied staples.

The anvil of a conventional linear stapling instrument may incorporate a relatively thick metal plate which is coined to form staple receiving pockets for the staples and for the opening or slot which accommodates the knife. The manufacture of an anvil via conventional coining processes is expensive considering the complexity of the machinery, and is also labor intensive. In addition, assembly of the anvil within the stapling instrument often requires delicate welding applications which are highly skill dependent often resulting in rejected units and associated extra expense.

### SUMMARY

Accordingly, the present disclosure is directed to an anvil, particularly, an anvil plate for use in a linear stapling instrument, which may be readily manufactured and incorporated in the anvil body of the stapling instrument. In accordance with one exemplary embodiment, an anvil for use with a linear stapling instrument includes an anvil body defining a longitudinal axis and an anvil plate. The anvil plate includes a base having a first side defining at least one staple forming pocket and a second side having a pair of outer beams extending along the longitudinal axis and a central beam disposed between the outer beams. The central beam includes at least one mounting tab, which is receivable within at least one corresponding tab receiving opening of the anvil body to mount the anvil plate relative to the anvil body.

In embodiments, the anvil body includes an anvil support column extending the length thereof. The anvil plate is secured relative to the anvil support column. In some embodiments, the central beam of the anvil plate includes first and second mounting tabs respectively disposed on first and second lateral sides of the central beam. In certain embodiments, the anvil body includes first and second anvil reinforcing ribs respectively positioned adjacent the first and second lateral sides of the central beam. The first and second anvil reinforcing ribs have first and second tab receiving recesses for respective reception of the first and second mounting tabs of the anvil plate in secured relation therewith.

In embodiments, the first and second lateral sides of the central beam are at least partially separated by a recess extending along the length of the central beam.

In other embodiments, the pair of outer beams of the anvil plate is positioned about a peripheral segment of opposed sides of the anvil support column. In some embodiments, one of the outer beams of the anvil plate includes a fixation tab, which is engageable with a corresponding recess in a respective side of the anvil support column to facilitate longitudinal fixation of the anvil plate relative to the anvil support column.

In certain embodiments, the anvil plate is formed by a stamping process.

In another exemplary embodiment, a surgical stapling instrument includes a cartridge body, a staple cartridge mounted relative to the cartridge body, an anvil body coupled to the cartridge body, an anvil plate mounted to the anvil body, and a firing mechanism supported by the cartridge body. The anvil plate includes a base having a first side defining at least one staple forming pocket in opposition to the staple cartridge and a second side. The second side has a pair of outer beams extending along the longitudinal axis and a central beam disposed between the outer beams. The central beam includes at least one mounting tab. The at least one mounting tab is receivable within at least one corresponding tab receiving opening of the anvil body to mount the anvil plate relative to the anvil body.

In embodiments, the stapling instrument includes an anvil support column extending along the anvil body and first and second anvil reinforcing ribs mounted to the anvil support column. The first and second anvil reinforcing ribs are respectively positioned adjacent first and second lateral sides of the central beam of the anvil plate. The first and second anvil reinforcing ribs have first and second tab receiving openings for respective reception of first and second mounting tabs of the anvil plate in secured relation therewith.

The anvil plate of the present disclosure may be readily incorporated within a surgical stapling instrument such as a linear stapling instrument capable of firing multiple rows of staples in connection with an open or an endoscopic surgical procedure. The anvil plate is formed via a stamping process which facilitates the manufacturing and assembly processes while also providing precise formation of the staple forming pockets.

Other features of the present disclosure will become more readily apparent from the following description.

### BRIEF DESCRIPTION OF THE DRAWINGS

Various embodiments of the presently disclosed surgical stapling instrument and associated anvil plate are described herein below with reference to the drawings, wherein:

FIG. 1 is a perspective view of an exemplary embodiment of the surgical stapling instrument having the anvil plate of the present disclosure;

FIG. 2 is an exploded perspective view of the surgical stapling instrument illustrating the anvil body with the anvil plate, the cartridge body, the clamping lever, the staple cartridge and the firing member;

FIG. 3 is an exploded perspective view of the anvil body illustrating the handle, the anvil support column, the anvil reinforcing ribs and the anvil plate;

FIGS. 4-5 are perspective views of the anvil plate;

FIGS. 6, 7 and 8 are cross-sectional views of the anvil plate taken along the respective lines 6-6, 7-7 and 8-8 of FIG. 5;

FIG. 9 is a perspective view illustrating assembly of the anvil plate relative to the anvil support column;

FIGS. 10, 11 and 12 are cross-sectional views taken along the respective lines 10-10, 11-11 and 12-12 of FIG. 9;

FIG. 13 is a cross-sectional view taken along the lines 13-13 of FIG. 9 illustrating the longitudinal fixation element of the anvil plate engaging the anvil support column;

FIG. 14 is an enlarged isolated view of the area of detail identified in FIG. 2 illustrating the anvil plate mounted to the anvil body; and

FIG. 15 is a second enlarged perspective view illustrating the anvil plate mounted to the anvil body.

### DETAILED DESCRIPTION OF EMBODIMENTS

The surgical stapling instrument and associated anvil plate of the present disclosure will now be described in detail with reference to the drawings in which like reference numerals designate identical or corresponding elements in each of the several views. In this description, the term “proximal” is used generally to refer to that portion of the device that is closer to a clinician, while the term “distal” is used generally to refer to that portion of the device that is farther from the clinician. In addition, the term clinician is used generally to refer to medical personnel including doctors, nurses, and support personnel.

The surgical stapling instrument of the present disclosure includes an anvil plate, a staple cartridge, an actuator or lever that is configured to move the anvil and the staple cartridge between a spaced position and a clamped position, and a firing mechanism for ejecting staples from the staple cartridge. The anvil plate is readily adaptable within the anvil body of the surgical stapling instrument. The anvil plate is formed via a stamping process which facilitates formation of the staple receiving pockets when compared to conventional technologies. In embodiments, the anvil plate is formed of a metal strip of stainless steel or titanium with minimal thickness relative to conventional anvils. In this regard, the staple receiving pockets may be precisely dimensioned via the stamping process, and cooperate with the ejected staples to establish a more stable connection of the joined tissue. The stamping process also creates structural elements which enhance attachment of the anvil plate to the anvil body, and removes the necessity of alternate or additional attachment methodologies such as welding, brazing, adhesives, etc.

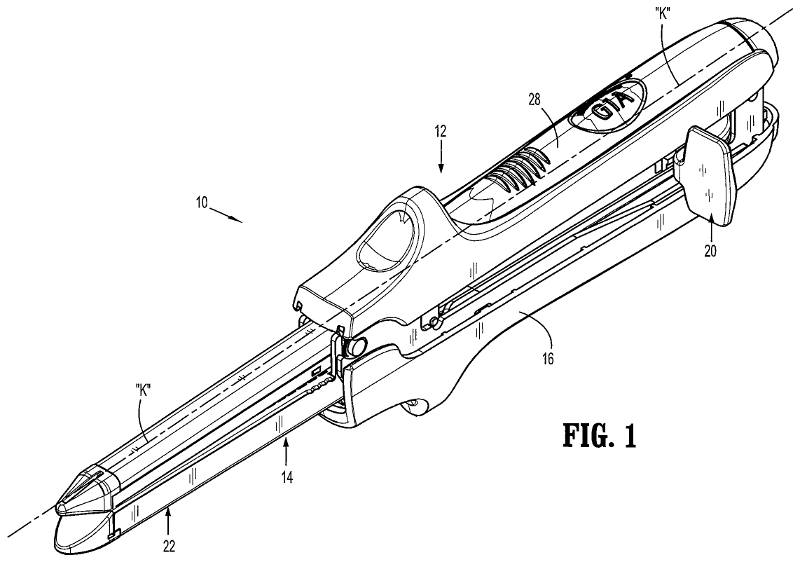

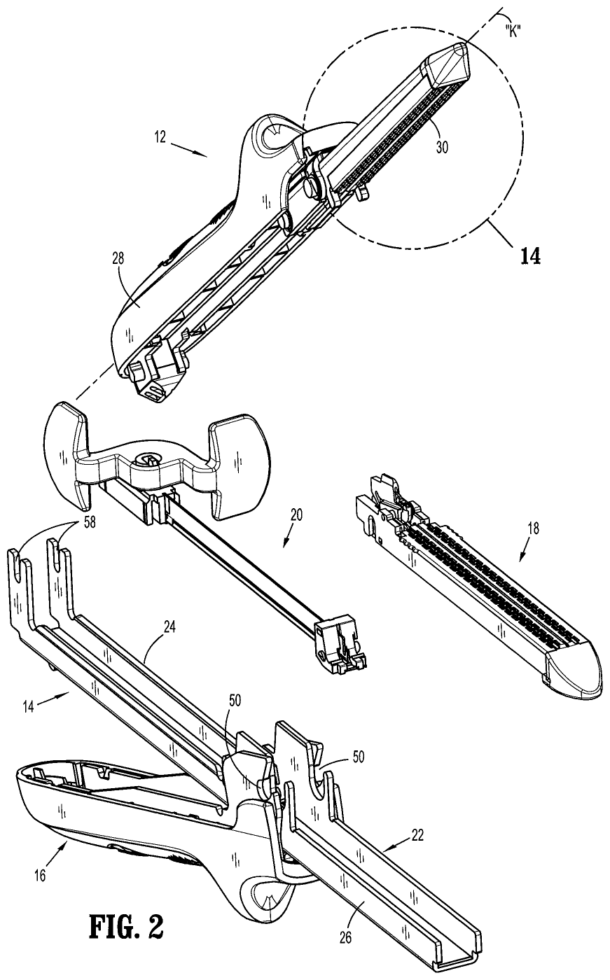

FIGS. 1-2 illustrate an exemplary embodiment of a surgical stapling instrument incorporating the anvil plate of the present disclosure and designated generally as stapling instrument 10. The stapling instrument 10 includes an anvil body 12 defining a longitudinal axis “k”, a cartridge body 14 supporting a clamping lever 16, a staple cartridge or single use loading unit (“SULU”) 18 mounted to the cartridge body 14 and a firing assembly 20. The cartridge body 14 includes a cartridge support column 22 having a proximal segment 24 and a distal segment 26. The proximal segment 24 of the cartridge support column 22 receives the firing assembly 20 and pivotally supports the clamping lever 16. The distal segment 26 of the cartridge support column 22 receives the staple cartridge 18. The anvil body 12 includes an anvil hand grip 28 and an anvil plate 30. The clamping lever 16 is pivotally supported on the cartridge body 14 and is engageable with the anvil body 12 such that movement of the clamping lever 16 from an unclamped position (FIG. 2) to a clamped position (FIG. 1) moves the anvil body 12 into close approximation with the cartridge body 14. In general, with the clamping lever 16 in the clamped position engaging tissue to be joined between the staple cartridge 18 and the anvil plate 30, the firing assembly 20 is actuated by advancing movement along the longitudinal axis “k” to eject a plurality of rows of staples from the staple cartridge 18 for engagement and deformation by the anvil plate 30. A knife (not shown) may traverse the staple cartridge 18 to sever tissue between the applied rows of staples.

For a detailed discussion of the construction and operation of an exemplary surgical stapling instrument, reference may be made to commonly assigned U.S. Patent U.S. Pat. No. 8,505,801 to Ehrenfels et al., the entire contents of which is incorporated herein by reference.

Referring now to FIG. 3, further details of the anvil body 12 will be discussed. The anvil body 12 includes the previously mentioned anvil hand grip 28, a saddle 32 which assists in pivotally mounting the anvil body 12 to the cartridge body 14, a general U-shaped anvil support column 34 extending along the length of the anvil hand grip 28 and along the anvil plate 30, and first and second anvil reinforcing ribs 36a, 36b secured within the anvil support column 34. In embodiments, the first and second reinforcing ribs 36a, 36b define apertures 38 at their proximal ends which are aligned with corresponding apertures 40 extending through the anvil support column 34. A pin 41 extends through the apertures 38, 40 to connect the components. The first reinforcing rib 36a includes a pair of first tab receiving recesses 42 (shown in phantom) and the second reinforcing rib 36b includes a single second centrally disposed tab receiving recess 44. The recesses 42, 44 assist in securing the anvil plate 30 relative to the anvil support column 34.

With continued reference to FIG. 3, the saddle 32 is secured within the anvil hand grip 28 and defines opposed mounting legs 46 with mounting bosses 48 which are secured to the outer surfaces respectively of the anvil support column 34 thereby securing the anvil support column 34 relative to the hand grip 28. The mounting bosses 48 couple with corresponding structure of the cartridge body 14, specifically, access channels 50 of the clamping lever 16 (FIG. 2) to enable movement of the anvil body 12 and the cartridge body 14 to the approximated condition. The anvil body 12 further includes a coupling pin 52 and pin mount 54 having a pin opening 56 for receiving the coupling pin 52. The coupling pin 52 is received within pin receiving slots 58 of the cartridge support column 22 (FIG. 2) to couple the components prior to use. The anvil body 12 also includes a nose 60 configured to be atraumatic to tissue.

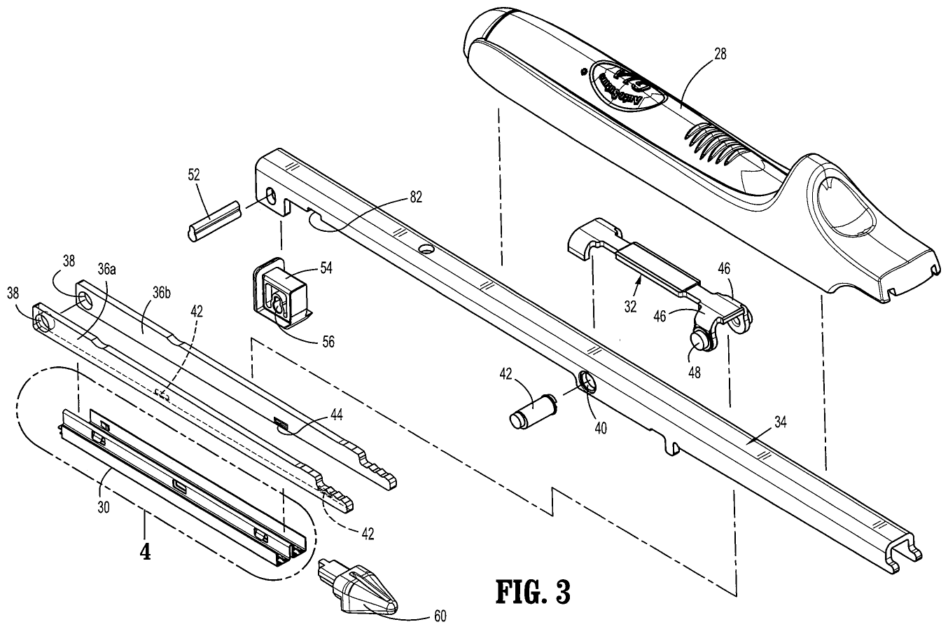

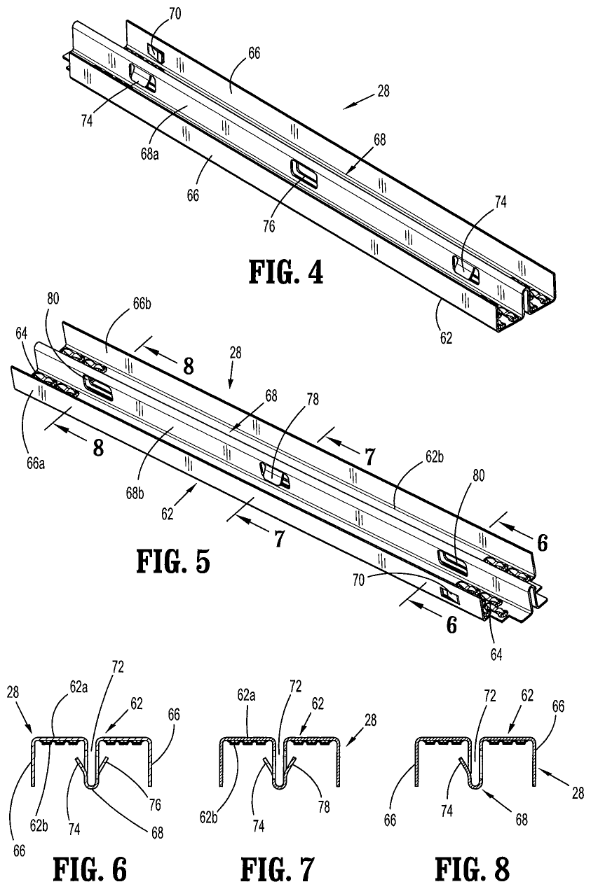

Referring now to FIG. 4-8, the anvil plate 30 of the anvil body 12 will be discussed. The anvil plate 30 is manufactured via a stamping process thereby avoiding costs and difficulties associated with conventional coining methodologies used in the manufacture of anvils for known stapling instruments. The stamping process utilized also provides mounting structure which facilitates securement of the anvil plate 30 relative to the anvil body 12. The anvil plate 30 includes a base 62 having staple forming pockets 64 on a first side 62a of the base 62 formed via the stamping process. (In FIGS. 4-8 only the staple forming pockets 64 at the ends of the anvil plate 30 are depicted for clarity purposes.) Extending from the second side 62b of the base 62 of the anvil plate 30 are opposed outer beams 66 and a central beam 68 which generally longitudinally bisects the two outer beams 66. One outer beam 66 has a longitudinal fixation tab 70 extending toward the central beam 68. The central beam 68 is generally U-shaped having first and second beam segments 68a, 68b on first and second lateral sides of the central beam 68 and separated by a longitudinal gap 72 running the length of the central beam 68. The longitudinal gap 72 accommodates the knife of the cartridge body 14 during actuation of the instrument.

As best depicted in FIG. 4, the first beam segment 68a of the central beam includes opposed first or end mounting tabs 74 adjacent the respective ends of the first beam segment 68a and a rectangular shaped aperture 76 generally at the midpoint of the first beam segment 68a. As depicted in FIG. 5, the second beam segment 68b of the central beam 68 includes a second or central mounting tab 78, and opposed rectangular shaped openings 80 adjacent the ends of the second beam segment 68b. The cross-sectional views of FIGS. 6-8 best illustrate the orientation of the end mounting tabs 74 of the first beam segment 68a and the central mounting tab 78 of the second beam segment 68b. As shown, each mounting tab 74, 78 includes an oblique hook segment (which may or may not be linear) that cooperates with the first and second tab receiving recesses 42, 44 of the first and second reinforcing ribs 36a, 36b to secure the anvil plate 30 relative to the anvil body 12 in opposition to the staple cartridge 18. The opposed outer beams 66 with the longitudinal fixation tab 70, the central beam 68, and the end and central mounting tabs 74, 78 are all formed during the coining process. In embodiments, the openings 76, 80 are a byproduct of the coining process and provide no other function other than permitting lateral movement of the respective adjacent mounting tabs 74, 78.

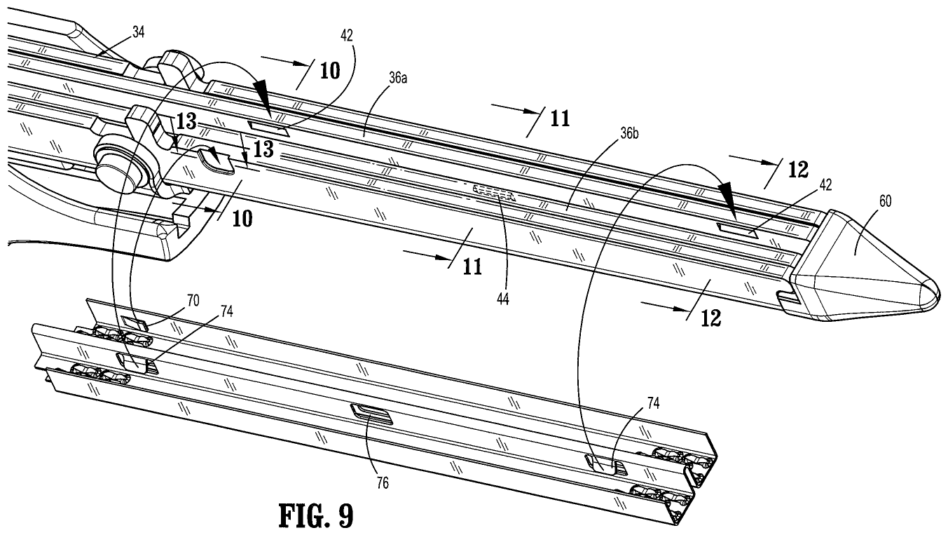

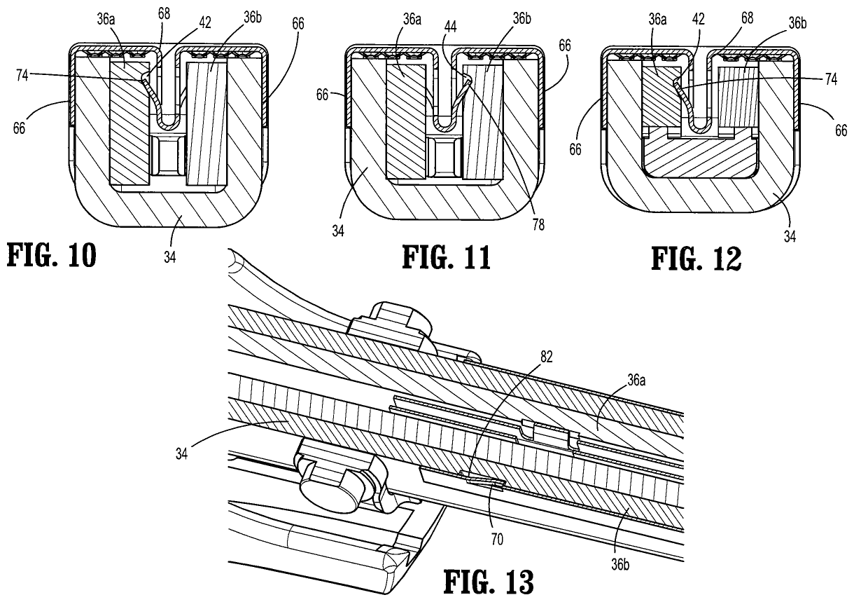

Referring now to FIGS. 9-12, mounting of the anvil plate 30 to the anvil support column 34 will be discussed. The first and second spaced reinforcing ribs 36a, 36b are secured within the anvil support column 34. As best depicted in FIG. 9, the end mounting tabs 74 of the first beam segment 68a are aligned with the opposed tab receiving recesses 42 of the first reinforcing rib 36a and the central mounting tab 78 of the second beam segment 68b is aligned with the centrally disposed tab receiving recess 44 of the second reinforcing rib 36b. The anvil plate 30 is rotated as shown onto the anvil support column 34. Upon positioning the anvil plate 30 about the anvil support column 34, the outer beams 66 are disposed over the outer surfaces of the anvil support column 34 while the central beam 68 is received within the space defined between the first and second reinforcing ribs 36a, 36b. Advancement of the anvil plate 30 onto the anvil support column 34 causes the opposed end mounting tabs 74 and the central mounting tab 78 to engage the respective end and central tab receiving recesses 42, 44 in snap relation thereby securing the anvil plate 30 to the anvil support column 34. In addition, with reference to FIG. 13, the fixation tab 70 is received within a fixation tab receiving recess 82 (FIG. 3) defined in the anvil support column 34 to restrict longitudinal movement of the anvil plate 30 relative to the anvil support column 34. Thus, the anvil plate 30 is secured to the anvil support column 34 through the mechanical connection of the mounting tabs 74, 78 of the anvil plate 30 with the tab receiving recesses 42, 44 of the first and second reinforcing ribs 36a, 36b without the use of additional welding, brazing or cementing technologies.

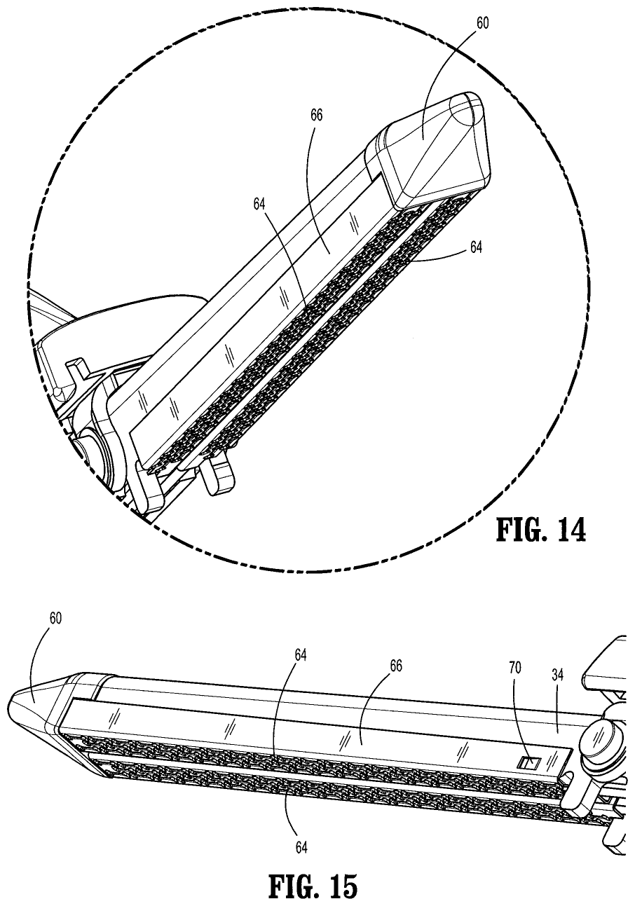

FIGS. 14-15 illustrate the anvil plate 30 mounted to the anvil support column 34 with the staple forming pockets 64 facing outwardly relative to the longitudinal axis “k” for positioning relative to the staple cartridge 18.

Although the presently disclosed surgical stapling instrument 10 is illustrated as an open-type linear surgical stapler, it is envisioned that the benefits of the present disclosure may be incorporated into a variety of different types of surgical stapling instruments including, e.g., endoscopic linear staplers. The benefits of the presently disclosed stapling instrument are particularly relevant to stapling devices having linear or curved cantilevered jaws.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. An anvil for use with a linear stapling instrument, comprising;
an anvil body defining a longitudinal axis; and
an anvil plate including a base having a first side defining at least one staple forming pocket and a second side having a pair of outer beams extending along the longitudinal axis and a central beam disposed between the outer beams, the central beam including at least one mounting tab, the at least one mounting tab receivable within at least one corresponding tab receiving opening of the anvil body to mount the anvil plate relative to the anvil body.

2. The anvil according to claim 1 wherein the anvil body includes an anvil support column extending the length thereof, the anvil plate being secured relative to the anvil support column.

3. The anvil according to claim 2 wherein the central beam of the anvil plate includes first and second mounting tabs respectively disposed on first and second lateral sides of the central beam.

4. The anvil according to claim 3 wherein the anvil body includes first and second anvil reinforcing ribs respectively positioned adjacent the first and second lateral sides of the central beam, the first and second anvil reinforcing ribs having first and second tab receiving recesses for respective reception of the first and second mounting tabs in secured relation therewith.

5. The anvil according to claim 1 wherein the first and second lateral sides of the central beam are at least partially separated by a recess extending along the length of the central beam.

6. The anvil according to claim 2 wherein the pair of outer beams of the anvil plate is positioned about a peripheral segment of opposed sides of the anvil support column.

7. The anvil according to claim 6 wherein one of the outer beams of the anvil plate includes a fixation tab, the fixation tab engageable with a corresponding recess in a respective side of the anvil support column to facilitate longitudinal fixation of the anvil plate relative to the anvil support column.

8. The anvil according to claim 1 wherein the anvil plate is formed by a stamping process.

9. A surgical stapling instrument comprising:
a cartridge body;
a staple cartridge mounted relative to the cartridge body;
an anvil body coupled to the cartridge body;
an anvil plate mounted to the anvil body, the anvil plate including a base having a first side defining at least one staple forming pocket in opposition to the staple cartridge and a second side, the second side having a pair of outer beams extending along the longitudinal axis and a central beam disposed between the outer beams, the central beam including at least one mounting tab, the at least one mounting tab receivable within at least one corresponding tab receiving opening of the anvil body to mount the anvil plate relative to the anvil body; and
a firing mechanism supported by the cartridge body.

10. The surgical stapling instrument according to claim 9 including
an anvil support column extending along the anvil body; and
first and second anvil reinforcing ribs mounted to the anvil support column, the first and second anvil reinforcing ribs respectively positioned adjacent first and second lateral sides of the central beam, the first and second anvil reinforcing ribs having first and second tab receiving openings for respective reception of first and second mounting tabs of the anvil plate in secured relation therewith.
